(19) United States
(12) Patent Application Publication
(10) Pub. No.: US 2025/0134947 A1
(43) Pub. Date: May 1, 2025

(54) TRI-, TETRA AND PENTAPEPTIDES, COMPOSITIONS THEREOF AND THEIR USE IN THE THERAPY OF PSORIASIS

(71) Applicant: ENUMITA AS, Tromsdalen (NO)

(72) Inventor: Ole-Jan IVERSEN, Tromsdalen (NO)

(21) Appl. No.: 18/706,116

(22) PCT Filed: Oct. 27, 2022
(86) PCT No.: PCT/NO2022/000006

§ 371 Date: Apr. 30, 2024

(30) Foreign Application Priority Data
Nov. 2, 2021 (NO) 20211318
Feb. 7, 2022 (NO) 20220171
Mar. 11, 2022 (NO) 20220313

(51) Int. Cl. A61K 38/06; A61K 8/64; A61K 9/00; A61K 38/07; A61K 45/06; A61P 17/06; A61Q 19/00

(52) U.S. Cl. CPC A61K 38/06; A61K 8/64; A61K 9/0014; A61K 38/07; A61K 45/06; A61P 17/06; A61Q 19/00

(57) ABSTRACT

The invention relates to a peptide for use in prophylaxis, prevention, inhibition and/or treatment of psoriasis of a mammal, wherein the peptide comprises amino acids joined by peptide bonds, and wherein the peptide comprises an amino acid sequence of from 3 to 5 amino acids comprising 2 acidic amino acids and proline (P) (Pro). The invention also relates to a pharmaceutical composition for use in prophylaxis, prevention, inhibition and/or treatment of psoriasis of a mammal, wherein the pharmaceutical composition comprises a peptide, wherein the peptide comprises amino acids joined by peptide bonds, and wherein the peptide comprises an amino acid sequence of from 3 to 5 amino acids comprising 2 acidic amino acids and proline (P) (Pro). The invention further relates to a composition for skin improvement, a cosmetic composition, a topical skin cream composition and a nutritional compositions, wherein said compositions comprise a peptide comprising amino acids joined by peptide bonds, wherein the peptide comprises an amino acid sequence of from 3 to 5 amino acids comprising 2 acidic amino acids and proline (P) (Pro).

### FIELD OF THE INVENTION

The present invention relates to a peptide and pharmaceutical, cosmetic, topical skin cream and nutritional compositions comprising the peptide. The pharmaceutical composition comprising the peptide according to the invention make it possible to prevent, inhibit and/or treat the inflammatory loop that contributes to chronicity of psoriasis and other inflammatory diseases, and hereby it can be used for profylaxis, prevention, inhibition and/or treatment of psoriasis and chronic inflammatory diseases. The cosmetic, topical skin cream and nutritional compositions comprising the peptide according to the invention can be used for improving the appearance or rejuvenating the appearance of skin and for improving the gastrointestinal regularity, health and overall well-being.

### BACKGROUND OF THE INVENTION

The immune system plays a crucial role in combating injurious agents, such as bacteria, viruses, cancer cells and toxins. Binding of specific antibodies to the agent (foreign antigen) causes cascade reactions and give rise to inflammatory reactions. When the agent is eliminated, the inflammation is down-regulated and the tissue will reverse to normal. Chronic inflammation might progress when the acute response cannot be resolved either because of the persistence of the injurious agent or because of the interference in the normal process of healing. An aberrant immune response occurs if antibodies attack the body's own healthy cells and tissues (self- or autoantigens) and leads to inflammation. These responses are categorized as an autoimmune reaction and underlie a wide range of clinical disorders. While autoimmune diseases are a pathologic state, autoimmunity nevertheless derives from the same mechanisms that underlie the normal immune response to foreign antigens.

It is disclosed in a scientific paper by Iversen O J, Lysvand H, Slupphaug G, “Pso p27, a SerpinB3/B4 derived protein, is most likely a common autoantigen in chronic inflammatory diseases”, Clinical Immunology 174 (2017), 10-17, that autoimmune diseases are characterized by chronic inflammatory reactions localized to an organ or organ system. They are caused by immunologic reactions toward self-antigens, causing formation of autoantibodies that mistakenly attack their own body. Psoriasis is a chronic inflammatory autoimmune skin disease in which a serpin-derived protein Pso p27 is an autoantigen.

It is further disclosed that Pso p27 is derived from the serpin molecules SerpinB3 and SerpinB4 through non-canonical cleavage by mast cell chymase. In psoriasis, Pso p27 is exclusively found in skin lesions and not in uninvolved skin. The serpins are cleaved into three fragments that remain associated as a Pso p27 complex increased tendency to form large aggregates compared to native SerpinB3/B4, and with immunogenic properties. The amount of Pso p27 is directly correlated to disease activity, and through formation of complement activating immune-complexes, Pso p27 contribute to the inflammation in the skin lesions. SerpinB3/B4 are expressed in skin fibroblasts and keratinocytes, but normally absent in mast cells. Over-expression of the serpins may be induced by inflammation and hypoxia. SerpinB3 and SerpinB4 are taken up in mast cells. Here the generation and subsequent release of Pso p27 aggregates promote an inflammatory loop that contributes to the chronicity of psoriasis.

It is further disclosed in this paper that Pso p27 and/or antibodies against Pso p27 have been demonstrated in the affected organs in various chronic inflammatory diseases such as psoriasis, atopic dermatitis, rheumatoid arthritis, ankylosing spondylitis, ulcerous colitis, Crohn's disease and sarcoidosis, indicating that Pso p27 complexes are common auto-antigen that contribute to the inflammation in chronic inflammatory diseases.

It can be concluded that the mechanism comprising Pso p27 and/or aggregates thereof, and the serpin molecules SerpinB3 and SerpinB4 disclosed above, is a common mechanism in chronic inflammatory diseases in general, including cancer, cf. Turato C, Pontisso P, “SerpinB3 (serpin peptidase inhibitor, clade B (ovalbumin) member 3)”, Atlas Genet Cytogenet Oncol Haemotol. 19 (3) (2015) 202-209; Walker M E, Hatfield J K, Brown M A, “New insight into the role of mast cells in autoimmunity: Evidence for a common role of action?”, Biochim. Biophys. Acta 1822 (2012) 57-65; and Greten F R, Grivennikov S I, “Inflammation and cancer: Triggers, mechanisms, and consequences”, Immunity. 2019; 51:27-41.

It would be desirable to be able to prevent, inhibit and/or treat the inflammatory loop that contributes to the chronicity of psoriasis and other chronic inflammatory diseases such as autoimmune diseases. It would also be desirable to provide an inhibitor, that may prevent and/or inhibit the cleavage reaction of serpin molecules SerpinB3/B4 and formation of Pso p27. It would also be desirable to provide a pharmaceutical composition which can be used for profylaxis, prevention, inhibition and/or treatment of psoriasis and other chronic inflammatory diseases such as autoimmune diseases and, more specifically, for profylaxis, prevention, inhibiting and/or treatment of chronic inflammatory reactions of autoimmune diseases and cancer and avoiding the use of immune-suppressing agents. It would also be desirable to provide a composition that may be used for improving the appearance or rejuvenating the appearance of skin and improving gastrointestinal regularity, health and overall well-being.

### SUMMARY OF THE INVENTION

It is an object of the present invention to provide a peptide and pharmaceutical composition comprising a peptide that can be used for profylaxis, prevention, inhibition and/or treatment of psoriasis.

It is another object of the present invention to provide a peptide and pharmaceutical composition comprising a peptide that can be used for profylaxis, prevention, inhibition and/or treatment of chronic inflammatory diseases.

By using the peptide and pharmaceutical composition comprising the peptide according to the invention, it is possible to prevent, inhibit and/or treat a chronic inflammation of a subject suffering from psoriasis or a chronic inflammatory disease which leads to reduced disease activity, either temporarily or permanently. Hereby the present invention may lead to a prophylactic treatment or reduction in chronic inflammation and associated organ dysfunction, organ lesions, milder symptoms, improvement in disease activity and/or increased life quality.

It is another object of the present invention to provide a peptide and cosmetic, topical skin cream and nutritional compositions comprising a peptide that can be used for improving the appearance or rejuvenating the appearance of skin and for improving gastrointestinal regularity, health and overall well-being.

Accordingly, in one aspect, the present invention relates to a peptide for use in profylaxis, prevention, inhibition and/or treatment of psoriasis of a mammal, wherein the peptide comprises amino acids joined by peptide bonds, and wherein the peptide comprises an amino acid sequence of from 3 to 5 amino acids which comprises 2 acidic amino acids and proline (P) (Pro). This may mean that the peptide could have any length, as long as 2 acidic amino acids and proline (P) (Pro) are within a distance of 3 to 5 amino acids.

In another aspect, the present invention relates to a peptide for use in profylaxis, prevention, inhibition and/or treatment of a chronic inflammatory disease of a mammal, wherein the peptide comprises amino acids joined by peptide bonds, and wherein the peptide comprises an amino acid sequence of from 3 to 5 amino acids which comprises 2 acidic amino acids and proline (P) (Pro). Also in this context, the peptide could have any length, as long as 2 acidic amino acids and proline (P) (Pro) are within a distance of 3 to 5 amino acids.

In another aspect, the present invention relates to a pharmaceutical composition for use in profylaxis, prevention, inhibition and/or treatment of psoriasis of a mammal, wherein the pharmaceutical composition comprises a peptide, wherein the peptide comprises amino acids joined by peptide bonds, and wherein the peptide comprises an amino acid sequence of from 3 to 5 amino acids which comprises 2 acidic amino acids and proline (P) (Pro).

In another aspect, the present invention relates to a pharmaceutical composition for use in profylaxis, prevention, inhibition and/or treatment of a chronic inflammatory disease of a mammal, wherein the pharmaceutical composition comprises a peptide, wherein the peptide comprises amino acids joined by peptide bonds, and wherein the peptide comprises an amino acid sequence of from 3 to 5 amino acids which comprises 2 acidic amino acids and proline (P) (Pro).

In another aspect, the present invention relates to a pharmaceutical composition for use in profylaxis, prevention, inhibition and/or treatment of psoriasis of a mammal, to be administered to the mammal, wherein the pharmaceutical composition comprises a peptide, wherein the peptide comprises amino acids joined by peptide bonds, and wherein the peptide comprises an amino acid sequence of from 3 to 5 amino acids which comprises 2 acidic amino acids and proline (P) (Pro).

In another aspect, the present invention relates to a pharmaceutical composition for use in profylaxis, prevention, inhibition and/or treatment of a chronic inflammatory disease of a mammal, to be administered to the mammal, wherein the pharmaceutical composition comprises a peptide, wherein the peptide comprises amino acids joined by peptide bonds, and wherein the peptide comprises an amino acid sequence of from 3 to 5 amino acids which comprises 2 acidic amino acids and proline (P) (Pro).

In another aspect, the present invention relates to a composition for skin improvement which comprises a peptide, wherein the peptide comprises amino acids joined by peptide bonds, and wherein the peptide comprises an amino acid sequence of from 3 to 5 amino acids which comprises 2 acidic amino acids and proline (P) (Pro).

In another aspect, the present invention relates to a cosmetic composition comprising a peptide, wherein the peptide comprises amino acids joined by peptide bonds, and wherein the peptide comprises an amino acid sequence of from 3 to 5 amino acids which comprises 2 acidic amino acids and proline (P) (Pro).

In another aspect, the present invention relates to a topical skin cream composition comprising a peptide, wherein the peptide comprises amino acids joined by peptide bonds, and wherein the peptide comprises an amino acid sequence of from 3 to 5 amino acids which comprises 2 acidic amino acids and proline (P) (Pro).

In yet another aspect, the present invention relates to a nutritional composition comprising a peptide, wherein the peptide comprises amino acids joined by peptide bonds, and wherein the peptide comprises an amino acid sequence of from 3 to 5 amino acids which comprises 2 acidic amino acids and proline (P) (Pro).

These and other objects and aspects of the invention will be described in further detail hereinafter.

### BRIEF DESCRIPTION OF THE DRAWING

FIG. 1 is a schematic illustration of the feedback mechanism for the chronicity in chronic inflammatory diseases (Iversen O J, Lysvand H, Slupphaug G, “Pso p27, a SerpinB3/B4 derived protein, is most likely a common autoantigen in chronic inflammatory diseases”, Clinical Immunology 174 (2017), 10-17).

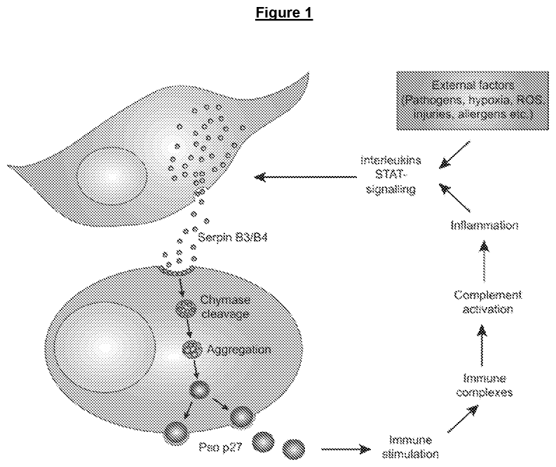

FIG. 2 shows the results obtained by SDS-gel-electrophoresis of tetrapeptides, indicating that the tetrapeptides according to the invention were effective in inhibiting the transformation of SerpinB3 to Pso p27 with chymase. The line to the right represents generation of Pso p27 in the absence of a tetrapeptide according to the invention.

FIG. 3 shows the results obtained by SDS-gel-electrophoresis of tripeptides, indicating that the tripeptides according to the invention were effective in inhibiting the transformation of SerpinB3 to Pso p27 with chymase. The line to the left of “DPE” represents generation of Pso p27 in the absence of a tripeptide according to the invention.

FIG. 4 shows a first row of photos of skin lesions in the form of eczema spots of a first volunteer test person suffering from psoriasis before treatment (photos to the left dated 19 Nov. 2021) and after treatment (photos to the right dated 1 Dec. 2021) with a first composition containing a tetrapeptide DDPK (Asp-Asp-Pro-Lys) (SEQ ID NO. 5) according to the invention by topical administration. For the purpose of control, FIG. 4 also shows a second row of photos of skin lesions in the form of eczema spots of the said first volunteer test person before treatment (photos to the left dated 19 Nov. 2021) and after treatment (photos to the right dated 1 Dec. 2021) with a second composition which was similar to the first composition but which did not contain the tetrapeptide DDPK (Asp-Asp-Pro-Lys) (SEQ ID NO. 5).

FIG. 5 shows a first row of photos of skin lesions in the form of eczema spots of a second volunteer test person suffering from psoriasis before treatment (photo to the left dated 19 Nov. 2021) and after treatment (photo to the right dated 1 Dec. 2021) with the said first composition containing the tetrapeptide DDPK (Asp-Asp-Pro-Lys) (SEQ ID NO. 5) according to the invention by topical administration. For the purpose of control, FIG. 5 also shows a second row of photos of skin lesions in the form of eczema spots of the second volunteer test person before treatment (photo to the left dated 19 Nov. 2021) and after treatment (photo to the right dated 1 Dec. 2021) with the said second composition which was similar to the first composition but which did not contain the tetrapeptide DDPK (Asp-Asp-Pro-Lys) (SEQ ID NO. 5).

### DETAILED DESCRIPTION OF THE INVENTION

According to the invention, there is provided a peptide for use in profylaxis, prevention, inhibition and/or treatment of psoriasis or a chronic inflammatory disease of a mammal. There is also provided a pharmaceutical composition for use in profylaxis, prevention, inhibition and/or treatment of psoriasis or a chronic inflammatory disease of a mammal, wherein the pharmaceutical composition comprises a peptide. Preferably, the pharmaceutical composition is to be administered to the mammal. There is also provided a composition for skin improvement which comprises a peptide as well as cosmetics, topical skin cream and nutritional compositions comprising a peptide. The term “compositions”, as used herein, is meant to include one or more or all of the pharmaceutical composition, composition for skin improvement, cosmetic composition, topical skin cream composition and nutritional composition of the invention, unless otherwise stated.

Peptides are short chains of amino acids linked or joined by peptide bonds. As defined by IUPAC, cf. https://www.genecorner.ugent.be/iupac.html, there is an amino acid code, and a three letter code, for each amino acid, wherein the following amino acid and three letter codes are used for the indicated amino acids: D (Asp)=Aspartic Acid, E (Glu)=Glutamic Acid, K (Lys)=Lysine, L (Leu)=Leucine, N (Asn)=Asparagine, P (Pro)=Proline, etc. As peptides comprise amino acids joined by peptide bonds in a certain sequence, peptides may be defined by the sequence in which the amino acids, or the codes thereof, are joined.

Preferably, the peptide for use according to the invention comprises 2 acidic amino acids and proline (P) (Pro) in an amino acid sequence of from 3 to 5 amino acids. According to the invention, the peptide comprises amino acids joined by peptide bonds, and an amino acid sequence of from 3 to 5 amino acids which comprises 2 acidic amino acids and proline (P) (Pro). The peptide may have any length, as long as 2 acidic amino acids and proline (P) (Pro)) are within a distance, or an amino acid sequence, of 3 to 5 amino acids. Preferably, the amino acid sequence comprises not more than 2 acidic amino acids. Further, the amino acid sequence preferably comprises 2 acidic amino acids and proline (P) (Pro), and optionally a neutral or basic amino acid. Preferably, the amino acid sequence comprises amino acids selected from aspartic acid (D) (Asp), glutamic acid (E) (Glu), lysine (K) (Lys), leucine (L) (Leu), asparagine (N) (Asn) and proline (P) (Pro). Preferably, the amino acid sequence comprises acidic amino acids which are selected from aspartic acid (D) (Asp) and/or glutamic acid (E) (Glu). Preferably, the neutral or basic amino acid is selected from lysine (K) (Lys), leucine (L) (Leu) and asparagine (N) (Asn).

According to the invention, the peptide may comprise an amino acid sequence of 3, 4 or 5 amino acids, for example and preferably being a tripeptide, tetrapeptide or pentapeptide, respectively, preferably the peptide contains a sequence of 3 or 4 amino acids, for example and preferably being a tripeptide or tetrapeptide. Preferably, the amino acid sequence is selected from the group consisting of DPE (Asp-Pro-Glu), EPD (Glu-Pro-Asp), DDP (Asp-Asp-Pro) and DEP (Asp-Glu-Pro), for example and preferably in the form of a tripeptide, as well as DPEN (Asp-Pro-Glu-Asn) (SEQ ID NO. 1), DPEL (Asp-Pro-Glu-Leu) (SEQ ID NO. 2), DLEP (Asp-Leu-Glu-Pro) (SEQ ID NO. 3), DDKP (Asp-Asp-Lys-Pro) (SEQ ID NO. 4), DDPK (Asp-Asp-Pro-Lys) (SEQ ID NO. 5), ENDP (Glu-Asn-Asp-Pro) (SEQ ID NO. 6), EDNP (Glu-Asp-Asn-Pro) (SEQ ID NO. 7), DEPN (Asp-Glu-Pro-Asn) (SEQ ID NO. 8), EDLP (Glu-Asp-Leu-Pro) (SEQ ID NO. 9), DEPL (Asp-Glu-Pro-Leu) (SEQ ID NO. 10), DELP (Asp-Glu-Leu-Pro) (SEQ ID NO. 11), DPNE (Asp-Pro-Asn-Glu) (SEQ ID NO. 12), DKPD (Asp-Lys-Pro-Asp) (SEQ ID NO. 13) and DPLE (Asp-Pro-Leu-Glu) (SEQ ID NO. 14), for example and preferably in the form of a tetrapeptide. According to the invention, the peptide comprises an amino acid sequence in which one or more amino acids may be in the form of a salt or an ion, e.g. a carboxylate ion. Examples of suitable salts of the peptide according to the invention include physiologically acceptable acid addition salts, e.g. salts with inorganic acids, e.g. hydrochloric acid, phosphoric acid, hydrobromic acid, sulfuric acid, as well as organic acids, e.g. acetic acid, formic acid, propionic acid, fumaric acid, maleic acid, succinic acid, tartaric acid, citric acid, malic acid, succinic acid, benzoic acid, methanesulfonic acid, benzenesulfonic acid and the like.

The peptides of the invention can be synthesized and/or are commercially available from, for example, (i) Biomatik Corporation, 4 Third Ave, Kitchener, Ontario, N2C 1N6, Canada, and (ii) Biomatik USA, LLC, 105-501 Silverside Road, Wilmington, Delaware, 19809, USA.

The invention relates to profylaxis, prevention, inhibition and/or treatment of psoriasis or a chronic inflammatory disease of a mammal. Inflammatory reactions are typically ongoing, lasting or persisting as long as infectious agents are present or damaged tissues are not healed. Chronic inflammation or autoimmune disease is usually referred to as an ongoing inflammation in the absence of infectious agents or damaged tissue. The term “chronic”, as used herein, means that the inflammatory disease may be ongoing, lasting or persisting for an extended period of time. The chronic inflammatory disease is typically ongoing, lasting or persisting for months, years or throughout life. The chronic inflammatory disease may show one or more symptoms which may vary over time, from mild to severe, which may come and go, e.g. periods of no symptoms alternating with attacks consisting of severe symptoms, and which may show a change or progression over time. The one or more symptoms may also vary from mammal to mammal, and from individual to individual, e.g. in human beings.

Psoriasis and chronic inflammatory diseases may affect and/or have a negative impact on the digestive system, tissues, joints, skin, respiratory system and organs. Psoriasis and chronic inflammatory diseases may show one or more symptoms. Examples of common symptoms of psoriasis and chronic inflammatory diseases include fatigue, body pain, joint stiffness, depression or anxiety, gastrointestinal complications (diarrhea or constipation), weight gain, weight loss, low grade fewer, and persistent infections, and the symptoms may vary from disease to disease. According to the invention, the peptide and pharmaceutical composition are suitable for use in profylaxis, prevention, inhibition and/or treatment of psoriasis or a chronic inflammatory disease of a mammal. Hereby one or more symptoms of psoriasis or the chronic inflammatory disease may be prevented, inhibited, reduced, relieved, eliminated or become milder, the one or more symptoms may be so affected at least temporarily, e.g. longer periods of no symptoms or symptoms so affected, and preferably permanently. Further, hereby one or more of the digestive system, tissues, joints, skin, respiratory system and organs of the mammal affected by psoriasis or the chronic inflammatory disease may show signs of improved function, less negative impact, reduced dysfunction and reduced lesions.

Examples of psoriasis that can be prophylactically treated, prevented, inhibited and/or treated by the peptide and pharmaceutical composition according to the present invention include guttate, inverse, erythrodermic and pustular psoriasis, and psoriatic arthritis (PsA). Examples of chronic inflammatory diseases that can be prophylactically treated, prevented, inhibited and/or treated by the peptide and pharmaceutical composition according to the present invention include autoimmune diseases, asthma, chronic obstructive pulmonary disease and cancer. Examples of autoimmune diseases include acquired hemophilia, acute motor axonal neuropathy, Addison's disease, adult-onset Still's disease, alopecia areata, ankylosing spondylitis, anti-glomerular basement membrane nephritis, anti-neutrophil cytoplasmic antibody-associated vasculitis, anti-N-methyl-D-aspartate receptor encephalitis, anti-sperm antibodies, antiphospholipid syndrome, antisynthetase syndrome, aplastic anemia, autoimmune angioedema, autoimmune encephalitis, autoimmune enteropathy, autoimmune gastritis, autoimmune hemophilia, autoimmune hepatitis, autoimmune inner ear disease, autoimmune lymphoproliferative syndrome, autoimmune neutropenia, autoimmune oophoritis, autoimmune orchitis, autoimmune pancreatitis, autoimmune polyendocrine syndrome type 1, autoimmune polyendocrine syndrome type 2, autoimmune polyendocrine syndrome type 3, autoimmune progesterone dermatitis, autoimmune retinopathy, auto-immune thrombocytopenia purpura, autoimmune thyroiditis, autoimmune urticaria, autoimmune uveitis, balo concentric sclerosis, Behcet's disease, Bickerstaff's encephalitis, brachial neuropathy (Parsonage-Turner syndrome), Bullous pemphigoid, celiac disease, chronic fatigue syndrome, chronic inflammatory demyelinating polyneuropathy, Churg-Strauss syndrome, cicatricial pemphigoid, Cogan syndrome, cold agglutinin disease, complex regional pain syndrome, CREST syndrome, Crohn's disease, cryptogenic organizing pneumonia, cutaneous lupus erythematosus, dermatitis herpetiformis, dermatomyositis, diabetes mellitus type 1, diffuse interstitial keratitis, discoid lupus erythematosus, endometriosis, enthesitis-related arthritis, eosinophilic esophagitis, eosinophilic fasciitis, epidermolysis bullosa acquisita, episcleritis, encephalopathy associated with autoimmune thyroid disease, erythema nodosum, essential mixed cryoglobulinemia, Evans syndrome, Felty syndrome, fibromyalgia, gestational pemphigoid, giant cell arteritis, goodpasture syndrome, Graves' disease, Graves ophthalmopathy, Guillain-Barre syndrome, Hashimoto's Encephalopathy, Hashimoto Thyroiditis, hidradenitis suppurativa, idiopathic dilated cardiomyopathy, idiopathic pulmonary fibrosis, IgA nephropathy, lgG4-related systemic disease, inclusion body myositis, inflammatory bowel diseases (IBD), intermediate uveitis, interstitial cystitis, juvenile arthritis, Kawasaki's disease, Lambert-Eaton myasthenic syndrome, leukocytoclastic vasculitis, lichen planus, lichen sclerosus, ligneous conjunctivitis, linear IgA disease, lupus nephritis, lupus vasculitis, Lyme disease (chronic), Ménière's disease, microscopic colitis, microscopic polyangiitis, mixed connective tissue disease, Mooren's ulcer morphea, Mucha-Habermann disease, multiple sclerosis, myasthenia gravis, myelin oligodendrocyte glycoprotein disease (MOG), myocarditis, myositis, narcolepsy with cataplexy, neuromyelitis optica, neuromyotonia opsoclonus myoclonus syndrome, optic neuritis, Ord's thyroiditis, palindromic rheumatism, paraneoplastic cerebellar degeneration, Parry Romberg syndrome, Parsonage-Turner syndrome, pediatric autoimmune neuropsychiatric disorder associated with streptococcus, pemphigus vulgaris, pernicious anemia, pityriasis lichenoides et varioliformis acuta, POEMS syndrome, polyarteritis nodosa, polymyalgia rheumatica, polymyositis postmyocardial infarction syndrome, postpericardiotomy syndrome, primary biliary cirrhosis, primary sclerosing cholangitis, psoriasis, including guttate, inverse, erythrodermic and pustular psoriasis, psoriatic arthritis (PsA), pure red cell aplasia, purpura rheumatica, pyoderma gangrenosum, Raynaud's phenomenon, reactive arthritis, relapsing polychondritis, restless leg syndrome, retinocochleocerebral vasculopathy, retroperitoneal fibrosis, rheumatic chorea, rheumatic fever, rheumatoid arthritis (RA), rheumatoid vasculitis, sarcoidosis, Schnitzler syndrome, scleritis, scleroderma, Sjogren's syndrome, stiff person syndrome, subacute bacterial endocarditis, Susac's syndrome, Sydenham chorea, sympathetic ophthalmia, systemic lupus erythematosus (SLE), systemic scleroderma, Takayasu arteritis, Tolosa-Hunt syndrome, transverse myelitis, ulcerative colitis (UC), undifferentiated connective tissue disease, urticaria, urticarial vasculitis, vasculitis, vitiligo, warm autoimmune hemolytic anemia and combinations thereof, including multiple autoimmune syndrome, i.e. the combination of at least three autoimmune diseases (MAS). The autoimmune disease is suitably psoriasis. Preferably, the use of the peptide or pharmaceutical composition is for one or more of improving skin function, improving skin elasticity, reducing skin dysfunction and reducing skin lesions.

Asthma is a long-term or chronic inflammatory disease of the airways of the lungs. Chronic obstructive pulmonary disease (COPD) is a chronic inflammatory disease that causes obstructed airflow from the lungs. Cancer is a disease caused by an uncontrolled division of abnormal cells in a part of the body of a mammal, and may be seen as a chronic inflammatory disease. Examples of cancer diseases of the invention include colon cancer and prostate cancer.

Preferably, by using the peptide and pharmaceutical composition in profylaxis, prevention, inhibition and/or treatment of psoriasis or a chronic inflammatory disease according to the invention, there is a prevention and/or inhibition of the cleavage reaction of serpin molecules SerpinB3/B4, reduced formation and/or release of Pso p27 and/or complexes thereof, reduced amount of Pso p27 and/or complexes thereof, or a combination thereof.

The present invention further relates to improving the appearance or rejuvenating the appearance of skin. The skin is a complex organ which extends over the entire body of a human and other mammals. Although there are different types of skin at different portions of the body, skin is generally composed of two main layers of tissue; epidermis and dermis, where the epidermis is the outermost layer and the dermis provides a solid support for the epidermis. The dermis contains blood vessels, nerve fibres and an acellular part called extracellular matrix. Changes in the skin, in particular the extracellular matrix, can lead to skin aging, reduced skin elasticity, skin atrophy, skin lesions or to other conditions. Preferably, the peptide and composition for skin improvement, cosmetic composition and topical skin cream composition according to the invention lead to improving the state and appearance of skin, rejuvenating the appearance of skin, reducing visible signs of skin aging and/or enhancing the restoration of skin, and may also lead or contribute to improving skin function and/or elasticity, and/or reducing skin lesions, e.g. eczema, itching (pruritus) and atrophie blanche.

The present invention further relates to improving gastrointestinal regularity, health and overall well-being. The gastrointestinal tract is essentially a tube that extends from the mouth to the anus. It has generally the same structure throughout. There is a hollow portion of the tube known as the lumen, a muscular layer in the middle, and a layer of epithelial cells. These layers are responsible for maintaining the mucosal integrity of the tract. There are three main functions of the gastrointestinal tract, including transportation, digestion, and absorption of food. The mucosal integrity of the gastrointestinal tract (mouth, esophagus, stomach, small intestine, and large intestine) and the functioning of its accessory organs (liver, pancreas, and gallbladder) are vital in maintaining health. There are many conditions that may affect the gastrointestinal tract and system, e.g. irregular condition and discomfort associated with flatulence, heartburn, upset stomach, nausea, bloating, diarrhea and abdominal pain. Preferably, the use of the peptide and nutritional composition according to the invention lead to enhanced gastrointestinal regularity, health and overall well-being, which may be observed in terms of milder and/or less frequent symptoms or conditions of flatulence, heartburn, upset stomach, nausea, bloating, diarrhea and abdominal pain. The nutritional composition of the invention may also lead or contribute to improving the appearance or rejuvenating the appearance of skin.

The mammal of the invention may be any mammal, including rodents, e.g. mice, rats, hamsters and guinea pigs, lagomorphs, e.g. rabbits and hares, cats, dogs, farm animals, e.g. pigs, sheep, goats, horses, cows, deer and mink, primates, e.g. apes and monkeys, and humans, preferably the mammal is a human.

According to the invention, the peptide and compositions may be administered or applied to said mammal by various routes, e.g. one or more of the nasal, ophthalmic, oral, enteral, intragastric, sublingual, buccal, rectal, topical, transdermal, inhalational, pulmonary, parenteral, intramuscular, intravenous, intraperitoneal, intraocular, subcutaneous and intraarticular administration or application.

The peptide and compositions comprising the peptide of the invention may be provided in any form that is suitable for the intended use or administration. The pharmaceutical composition of the invention may be administered by any of the routes described above. The composition for skin improvement, cosmetic composition and topical skin cream composition of the invention are preferably used by topical application. The nutritional composition is preferably used by oral application.

The composition for skin improvement and cosmetic composition of the invention are preferably provided in the following forms or products: creams, ointments, gels, lotions, sprays, powders, aerosols, emollients, liniments and drops, preferably skin creams, skin ointments, skin gels, skin lotions, skin sprays, skin powders, skin aerosols, skin emollients, skin liniments and skin drops. The products may be used as they are or may be contained in or applied to a self-standing cosmetic sheet, e.g. as a sheet or face mask. Further examples of suitable products include topical skin cream compositions comprising the peptide according to the invention.

The present invention further relates to a topical skin cream composition comprising the peptide of the invention, i.e., a cream composition that is suitable for topical application on the skin. The topical skin cream composition of the invention may contain one or more of the suitable conventional additives and excipients described below and/or one or more pharma-ceutically active components described below. Usually, the topical skin cream composition of the invention contains more than 20% by weight of water and volatiles, and less than 50% by weight of hydrocarbons, waxes or macromolecules, e.g. polyethylene glycol, as a vehicle for external skin application.

The nutritional composition of the invention is preferably selected from the group consisting of food and beverage products. Examples of suitable food and beverage products include juice drinks, dairy drinks, powdered drinks, sports drinks, mineral water, soy beverages, hot chocolate, malt drinks, biscuits, bread, crackers, confectioneries, chocolate, chewing-gum, butters, margarines, spreads, yoghurts, breakfast cereals, snack bars, meal replacements, protein powders, desserts, medical nutrition feeds and nutritional supplements.

The pharmaceutical composition of the invention may be provided in the forms of pills, tablets (coated or uncoated), hard or soft capsules, dragees, lozenges, oral solutions, suspensions and dispersions, syrups or sterile parenteral preparations as well as in the forms described above for the other compositions of the invention.

In addition to the peptide, the compositions of the invention may also comprise one or more pharmaceutically inactive or acceptable components such as conventional additives, e.g. carriers, excipients or diluents, and/or one or more pharmaceutically active components.

Examples of suitable conventional additives and excipients that may be used according to the invention include inert diluents, e.g. carbonates, e.g. calcium carbonate and sodium carbonate, lactose, phosphates, e.g. calcium phosphate and sodium phosphate, granulating and disintegrating agents, e.g. corn starch or alginic acid, binding agents, e.g. starch, gelatine or acacia, effervescents, lubricating agents, e.g. magnesium stearate, stearic acid or talc, solvents, e.g. organic solvents, water and aqueous electrolyte solutions, preservatives, hydrocarbons, polymers or macromolecules, e.g. polyethylene glycols, chelating agents, effervescing agents, natural or artificial sweeteners, flavouring agents, colouring agents, taste masking agents, acidulants, emulsifiers, co-emulsifiers, thickening agents, suspending agents, dispersing or wetting agents, antioxidants, abrasive agents, absorbent powders, adhesion promoters, antacid agents, anti-cracking agents, anti-cellulite agents, anti-stretch mark agents, anti-dandruff agents, anti-foam agents, antiseptic agent, antistatic agent, barrier agents, binding agents, bio-adhesive agents, botanical agents, botanical extracts, biological additives, buffer agents, bulking agents, calcium sequestering agents, calming agents, carrier agents, cleansing agents, colorants, conditioning agents, controlled release agents, cooling agents, coupling agents, curative agents, denaturants, deodorant agents, depilatory agents, desquamating agent, detergents, disinfectants, dye stabilizers, dermatologically acceptable carriers, oils, emollients, fibers, film formers, fixatives, flavors, foam booster, foam stabilizer, foaming agent, fragrance, free radicals scavenger, macromolecules, hair beaching agents, hair growth promoters, hair colorants, hair conditioning agents, hair-set polymers, humectants, hydrophobic agents, hyaluronic acid stimulating agents, keratolytic agents, lathering agents, lipolytic agents, lubricants, make-up agents, moisture barrier agents, moisturizers, muco-adhesive agents, muscle relaxants, neutralizers, odor-masking agents, oil absorbent agents, ointment base agents, opacifiers, oxidants, oxygen carriers, pearlant agents, perfumes, perfume solvents, perfume stabilizers, pigments, plant extracts, plant root extracts, plant seed extracts, plant oils, plasticizers, polish agents, polymers, polymer film formers, preservative agents, propellants, reducing agents, re-fatting agents, scrub agents, skin barrier agents, skin calming agents, skin clarifiers, skin cleansers, skin conditioning agent, skin exfoliating agents, skin peeling agents, skin lightening agents, skin bleaching agents, skin protectant agents, skin smoothing agents, skin calming agents, skin soothing agents, solubilizers, sun protection factors, spreading agents, stabilizers, sunless tanning agents, sunscreen agents, e.g. sunscreen UVA, sunscreen UVB and broad-band sunscreen, surfactants, tanning accelerators, toners, tonic agents, topical delivery systems, viscosity stabilizers, waxes, whitening agents, exfoliants, and the like.

Examples of suitable pharmaceutically active components that may be used according to the invention include vitamins, e.g. vitamin A, vitamin B, vitamin C, vitamin D, vitamin E and vitamin K; anticoagulants, e.g. acetylsalicylic acid and COX-2 inhibitors; antithrombotics, fibrinolytic (thrombolytic) agents, antihypertensives, diuretics, antianginals, hypolipidemic agents, fibrates, beta-blockers, ACE inhibitors, cardiac glycosides, phosphodiesterase inhibitors, antiarrhythmics, calcium antagonists, polyphenols, phytosterols, agents modulating cell differentiation, agents modulating cell proliferation, agents stimulating synthesis of dermal or epidermal macromolecules, agents preventing degradation of dermal or epidermal macromolecules, agents acting on microcirculation, agents acting on skin barrier, agents acting on energy metabolism of cells, antimicrobial sequestering agents, analgesic agents, anti-acne agents, anti-skin aging agents, anti-skin wrinkle agents, anti-atrophy agents, anti-androgen agent, anti-bacterial agents, anti-skin scar agents, anti-seborrheic agents, anti-fungal agents, anti-histamine agents, anti-inflammatory agents, anti-irritant agents, anti-microbial agents, anti-mite agents, antibiotic agents, antiviral agents, anti-glycation agents, anti-neoplastic agents, anti-cancer agents, anti-eczema agents, antiperspirants, anti-pruriginous agents, anti-pruritic agents, astringents, α-adrenergic receptor agonists, circulatory stimulant agents, collagen stimulating agents, extracellular matrix stimulating agents, enzymes, coenzymes, enzymatic inhibitors, fungicides, analgesics, cytostatic drugs, and the like.

Preferably, the peptide or compositions of the invention is to be administered or applied to said mammal in an effective amount, which amount may vary depending on the type of administration or application, chronic inflammatory disease, composition and mammal.

### EXAMPLES

The invention is further illustrated in the following examples which, however, are not intended to limit the same. Parts and % relate to parts by weight and % by weight, respectively, and all suspensions are aqueous, unless otherwise stated.

### Example 1

In this Example, peptides were tested in order to find out whether they are able to prevent and/or inhibit the formation of Pso p27. The peptides were purchased from Biomatik Corporation, 4 Third Ave, Kitchener, Ontario, N2C 1N6, Canada, and analyzed with respect to prevention and/or inhibiting potential. The following methods were used:

### Synthesis SerpinB3

Expression vector pMCSG7 Serpin B3 (Clone ID: HsCD00343153) was used. Recombinant SerpinB3 was expressed in Escherichia coli BL21 (DE3) RIPL (Stratagene) and purified as described by Lysvand H, Helland R, Hagen L, Slupphaug G, Iversen OJ., “Psoriasis pathogenesis-Pso p27 is generated from SCCA 1 with chymase”, Biochim. Biophys. Acta 1842 (2014) 734-738.

### Test of Peptides

SerpinB3 solved in phosphate-buffered saline (PBS) (2 μg in 35 μl PBS) was incubated with a tripeptide or tetrapeptide 5 μl (10 mg/ml) for 1 hour at 37C°. 1 μl chymase was added and the mixture was incubated over night at 37C°. The solution was analyzed by SDS-gel-electrophoresis.

### SDS-Gel-Electrophoresis

SDS-gel-electrophoresis was performed as described by Iversen O J, Lysvand H, Hagen L., “The autoantigen Pso p27: a post-translational modification of SCCA molecules”, Autoimmunity 44 (2011) 229-234.

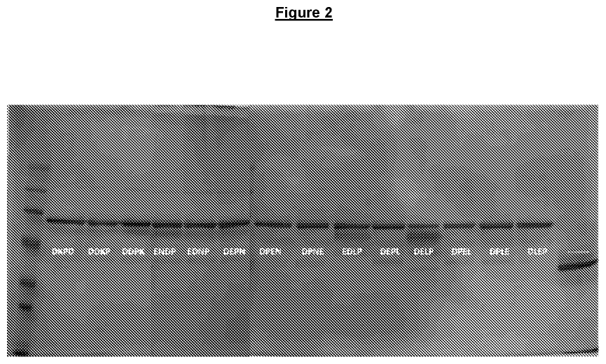

FIG. 2 shows the results obtained by SDS-gel-electrophoresis of tetrapeptides. As is evident from FIG. 2, tetrapeptides according to the invention were effective in inhibiting the transformation of SerpinB3 to Pso p27 with chymase. The line to the right represents generation of Pso p27 in the absence of a tetrapeptide according to the invention.

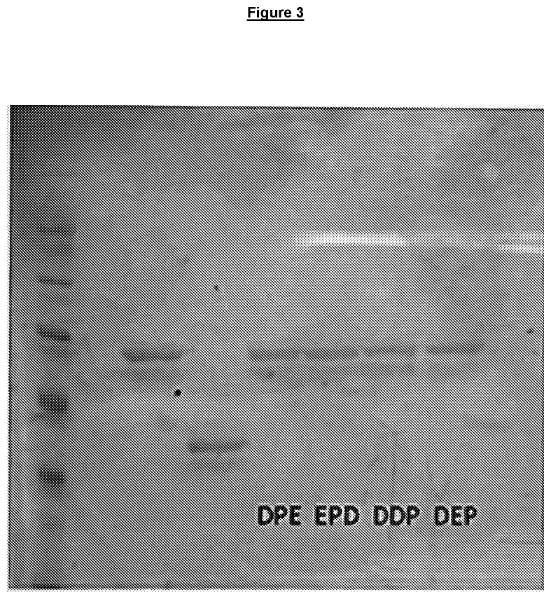

FIG. 3 shows the results obtained by SDS-gel-electrophoresis of tripeptides. As is evident from FIG. 3, tripeptides according to the invention were effective in inhibiting the transformation of SerpinB3 to Pso p27 with chymase. The line to the left of “DPE” represents generation of Pso p27 in the absence of a tripeptide according to the invention.

Tripeptides with an amino acid sequence of DPE (Asp-Pro-Glu), EPD (Glu-Pro-Asp), DDP (Asp-Asp-Pro) and DEP (Asp-Glu-Pro) as well as tetrapeptides with an amino acid sequence of DPEN (Asp-Pro-Glu-Asn) (SEQ ID NO. 1), DPEL (Asp-Pro-Glu-Leu) (SEQ ID NO. 2), DLEP (Asp-Leu-Glu-Pro) (SEQ ID NO. 3), DDKP (Asp-Asp-Lys-Pro) (SEQ ID NO. 4), DDPK (Asp-Asp-Pro-Lys) (SEQ ID NO. 5), ENDP (Glu-Asn-Asp-Pro) (SEQ ID NO. 6), EDNP (Glu-Asp-Asn-Pro) (SEQ ID NO. 7), DEPN (Asp-Glu-Pro-Asn) (SEQ ID NO. 8), EDLP (Glu-Asp-Leu-Pro) (SEQ ID NO. 9), DEPL (Asp-Glu-Pro-Leu) (SEQ ID NO. 10), DELP (Asp-Glu-Leu-Pro) (SEQ ID NO. 11), DPNE (Asp-Pro-Asn-Glu) (SEQ ID NO. 12), DKPD (Asp-Lys-Pro-Asp) (SEQ ID NO. 13) and DPLE (Asp-Pro-Leu-Glu) (SEQ ID NO. 14) were tested and found to be effective in prevention and/or inhibition of the cleavage reaction of the serpin molecules SerpinB3/B4, and/or prevention and/or inhibition of the formation of Pso p27.

Based on these results, it can be concluded that the peptides for use according to the invention, which comprise an amino acid sequence of from 3 to 5 amino acids which comprises 2 acidic amino acids and proline (P) (Pro), are suitable for use in profylaxis, prevention, inhibition and/or treatment of psoriasis or a chronic inflammatory disease of a mammal.

### Example 2

A first composition according to the invention was prepared by mixing the tetrapeptide DDPK (Asp-Asp-Pro-Lys) (SEQ ID NO. 5) with a commercial moisturizer. The resulting composition had a content of DDPK (Asp-Asp-Pro-Lys) (SEQ ID NO. 5) of 1.3% by weight. A second composition was provided as a reference to be used for control purposes. The second composition was based on the same commercial moisturizer but did not contain any tetrapeptide.

Two test persons suffering from psoriasis, skin dysfunction and skin lesions in the form of eczema spots, 35-50 years old, volunteered for tests the purpose of which was to find out whether treatment of their skin lesions with the tetrapeptide DDPK (Asp-Asp-Pro-Lys) (SEQ ID NO. 5) would have any effect. The volunteer test persons were subjected to treatment by topical administration on their skin lesions with the first composition according to the invention. As a control, the same volunteer test persons were subjected to treatment by topical administration on their skin lesions with the second composition used as a reference.

Photos of the skin lesions were taken before the treatment and twelve days after the treatment with the compositions.

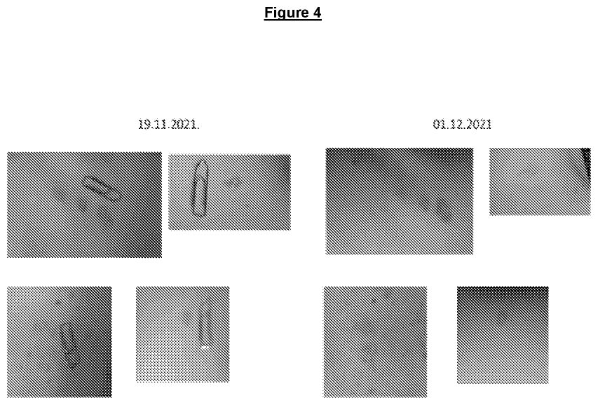

FIG. 4 shows a first or upper row of photos of skin lesions of the first volunteer test person before treatment (photos to the left dated 19 Nov. 2021) and after treatment (photos to the right dated 1 Dec. 2021) with the first composition containing DDPK (Asp-Asp-Pro-Lys) (SEQ ID NO. 5) according to the invention. For the purpose of control, FIG. 4 also shows a second or lower row of photos of skin lesions of the first volunteer test person before treatment (photos to the left dated 19 Nov. 2021) and after treatment (photos to the right dated 1 Dec. 2021) with the second composition which was similar to the first composition but which did not contain the tetrapeptide DDPK (Asp-Asp-Pro-Lys) (SEQ ID NO. 5).

It is evident from the photos of FIG. 4 that the treatment with a peptide and composition according to the invention resulted in improved appearance of the skin, reduced visible signs of skin lesions and enhanced the restoration of the skin over the treatment with a composition that did not contain such a peptide.

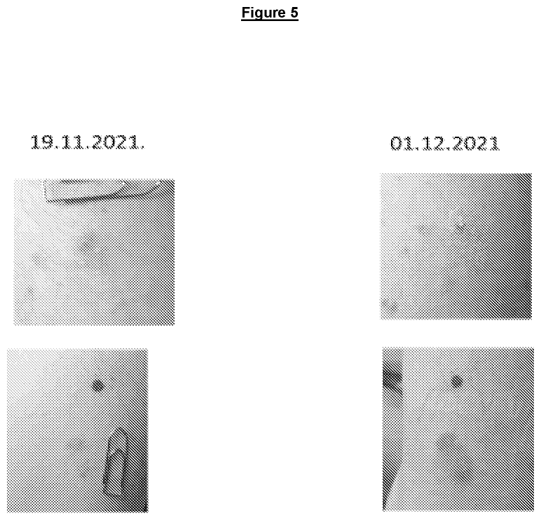

FIG. 5 shows a first or upper row of photos of skin lesions of the second volunteer test person before treatment (photo to the left dated 19 Nov. 2021) and after treatment (photo to the right dated 1 Dec. 2021) with the first composition containing DDPK (Asp-Asp-Pro-Lys) (SEQ ID NO. 5) according to the invention. For the purpose of control, FIG. 5 also shows a second or lower row of photos of skin lesions of the second volunteer test person before treatment (photo to the left dated 19 Nov. 2021) and after treatment (photo to the right dated 1 Dec. 2021) with the second composition which was similar to the first composition but which did not contain the tetrapeptide DDPK (Asp-Asp-Pro-Lys) (SEQ ID NO. 5).

Similarly, it is evident also from the photos of FIG. 5 that the treatment with a peptide and composition according to the invention resulted in improved appearance of the skin, reduced visible signs of skin lesions and enhanced the restoration of the skin over the treatment with a composition that did not contain such a peptide.

1. Peptide for use in profylaxis, prevention, inhibition and/or treatment of psoriasis of a mammal, wherein the peptide comprises amino acids joined by peptide bonds, and wherein the peptide comprises an amino acid sequence of from 3 to 5 amino acids comprising 2 acidic amino acids and proline (P) (Pro).

2. The peptide of claim 1, wherein the peptide comprises a pharmaceutical composition for use in profylaxis, prevention, inhibition and/or treatment of psoriasis of a mammal.

3. The peptide of claim 1, wherein the peptide comprises a composition for skin improvement.

4. Cosmetic composition comprising a peptide comprising amino acids joined by peptide bonds, wherein the peptide comprises an amino acid sequence of from 3 to 5 amino acids comprising 2 acidic amino acids and proline (P) (Pro), preferably the cosmetic composition is selected from the group consisting of creams, ointments, gels, lotions, sprays, powders, aerosols, emollients, liniments and drops.

5. The peptide of claim 1, wherein the peptide comprising a topical skin cream composition.

6. Nutritional composition comprising a peptide comprising amino acids joined by peptide bonds, wherein the peptide comprises an amino acid sequence of from 3 to 5 amino acids comprising 2 acidic amino acids and proline (P) (Pro), preferably the nutritional composition is selected from the group consisting of food and beverage products.

7. The peptide for use according to claim 1, wherein the amino acid sequence comprises not more than 2 acidic amino acids, preferably the amino acid sequence comprises 2 acidic amino acids, proline (P) (Pro) and optionally a neutral or basic amino acid, or 2 acidic amino acids, proline (P) (Pro) and a neutral or basic amino acid.

8. The peptide for use according to claim 1, wherein the acidic amino acids are selected from the group consisting of aspartic acid (D) (Asp) and glutamic acid (E) (Glu).

9. The peptide for use according to claim 1, wherein the neutral or basic amino acid is selected from the group consisting of lysine (K) (Lys), leucine (L) (Leu) and asparagine (N) (Asn).

10. The peptide for use according to claim 1, wherein the peptide is selected from the group consisting of tripeptides, tetrapeptides and pentapeptides, preferably the peptide is a tripeptide or tetrapeptide.

11. The peptide for use according to claim 1, wherein the amino acid sequence is selected from the group consisting of DPE (Asp-Pro-Glu), EPD (Glu-Pro-Asp), DDP (Asp-Asp-Pro), DEP (Asp-Glu-Pro), DPEN (Asp-Pro-Glu-Asn) (SEQ ID NO. 1), DPEL (Asp-Pro-Glu-Leu) (SEQ ID NO. 2), DLEP (Asp-Leu-Glu-Pro) (SEQ ID NO. 3), DDKP (Asp-Asp-Lys-Pro) (SEQ ID NO. 4), DDPK (Asp-Asp-Pro-Lys) (SEQ ID NO. 5), ENDP (Glu-Asn-Asp-Pro) (SEQ ID NO. 6), EDNP (Glu-Asp-Asn-Pro) (SEQ ID NO. 7), DEPN (Asp-Glu-Pro-Asn) (SEQ ID NO. 8), EDLP (Glu-Asp-Leu-Pro) (SEQ ID NO. 9), DEPL (Asp-Glu-Pro-Leu) (SEQ ID NO. 10), DELP (Asp-Glu-Leu-Pro) (SEQ ID NO. 11), DPNE (Asp-Pro-Asn-Glu) (SEQ ID NO. 12), DKPD (Asp-Lys-Pro-Asp) (SEQ ID NO. 13) and DPLE (Asp-Pro-Leu-Glu) (SEQ ID NO. 14).

12. The peptide for use according to claim 1, wherein the peptide comprises an amino acid sequence in which one or more amino acids is in the form of a salt or an ion.

13. The peptide for use according to claim 1, wherein the composition further comprises one or more additional pharmaceutically active component, preferably said one or more additional pharmaceutically active component is selected from the group consisting of vitamins, e.g. vitamin A, vitamin B, vitamin C, vitamin D, vitamin E and vitamin K; anticoagulants, e.g. acetylsalicylic acid and COX-2 inhibitors; antithrombotics, fibrinolytic (thrombolytic) agents, antihypertensives, diuretics, antianginals, hypolipidemic agents, fibrates, beta-blockers, ACE inhibitors, cardiac glycosides, phosphodiesterase inhibitors, antiarrhythmics, calcium antagonists, polyphenols, phytosterols, agents modulating cell differentiation, agents modulating cell proliferation, agents stimulating synthesis of dermal or epidermal macromolecules, agents preventing degradation of dermal or epidermal macromolecules, agents acting on microcirculation, agents acting on skin barrier, agents acting on energy metabolism of cells, antimicrobial sequestering agents, anti-acne agents, anti-skin aging agents, anti-skin wrinkle agents, anti-atrophy agents, anti-androgen agent, anti-bacterial agents, anti-skin scar agents, anti-seborrheic agents, anti-fungal agents, anti-histamine agents, anti-inflammatory agents, anti-irritant agents, anti-microbial agents, anti-mite agents, antibiotic agents, antiviral agents, anti-glycation agents, anti-neoplastic agents, anti-cancer agents, anti-eczema agents, antiperspirants, anti-pruriginous agents, anti-pruritic agents, astringents, α-adrenergic receptor agonists, circulatory stimulant agents, collagen stimulating agents, extracellular matrix stimulating agents, enzymes, coenzymes, enzymatic inhibitors, fungicides, analgesics and cytostatic drugs.

14. The peptide for use according to claim 1, wherein the composition further comprises one or more pharmaceutically inactive or acceptable components or conventional additives, preferably said one or more pharmaceutically inactive or acceptable components or conventional additives are selected from the group consisting of inert diluents, e.g. carbonates, e.g. calcium carbonate and sodium carbonate, lactose, phosphates, e.g. calcium phosphate and sodium phosphate, granulating and disintegrating agents, e.g. corn starch or alginic acid, binding agents, e.g. starch, gelatine or acacia, effervescents, lubricating agents, e.g. magnesium stearate, stearic acid or talc, solvents, e.g. organic solvents, water and aqueous electrolyte solutions, preservatives, hydrocarbons, polymers or macromolecules, e.g. polyethylene glycols, chelating agents, effervescing agents, natural or artificial sweeteners, flavouring agents, colouring agents, taste masking agents, acidulants, emulsifiers, co-emulsifiers, thickening agents, suspending agents, dispersing or wetting agents, antioxidants, abrasive agents, absorbent powders, adhesion promoters, antacid agents, anti-cracking agents, anti-cellulite agents, anti-stretch mark agents, anti-dandruff agents, anti-foam agents, antiseptic agent, antistatic agent, barrier agents, binding agents, bio-adhesive agents, botanical agents, botanical extracts, biological additives, buffer agents, bulking agents, calcium sequestering agents, calming agents, carrier agents, cleansing agents, colorants, conditioning agents, controlled release agents, cooling agents, coupling agents, curative agents, denaturants, deodorant agents, depilatory agents, desquamating agent, detergents, disinfectants, dye stabilizers, dermatologically acceptable carriers, oils, emollients, fibers, film formers, fixatives, flavors, foam booster, foam stabilizer, foaming agent, fragrance, free radicals scavenger, macromolecules, hair beaching agents, hair growth promoters, hair colorants, hair conditioning agents, hair-set polymers, humectants, hydrophobic agents, hyaluronic acid stimulating agents, keratolytic agents, lathering agents, lipolytic agents, lubricants, make-up agents, moisture barrier agents, moisturizers, muco-adhesive agents, muscle relaxants, neutralizers, odor-masking agents, oil absorbent agents, ointment base agents, opacifiers, oxidants, oxygen carriers, pearlant agents, perfumes, perfume solvents, perfume stabilizers, pigments, plant extracts, plant root extracts, plant seed extracts, plant oils, plasticizers, polish agents, polymers, polymer film formers, preservative agents, propellants, reducing agents, re-fatting agents, scrub agents, skin barrier agents, skin calming agents, skin clarifiers, skin cleansers, skin conditioning agent, skin exfoliating agents, skin peeling agents, skin lightening agents, skin bleaching agents, skin protectant agents, skin smoothing agents, skin calming agents, skin soothing agents, solubilizers, sun protection factors, spreading agents, stabilizers, sunless tanning agents, sunscreen agents, e.g. sunscreen UVA, sunscreen UVB and broad-band sunscreen, surfactants, tanning accelerators, toners, tonic agents, topical delivery systems, viscosity stabilizers, waxes, whitening agents, exfoliants, and the like.

15. The peptide for use according to claim 1, wherein the mammal is a human.


SEQUENCE LISTING (14 sequences)

SEQ ID NO: 1 - protein, 4 aa, synthetic construct
DPEN

SEQ ID NO: 2 - protein, 4 aa, synthetic construct
DPEL

SEQ ID NO: 3 - protein, 4 aa, synthetic construct
DLEP

SEQ ID NO: 4 - protein, 4 aa, synthetic construct
DDKP

SEQ ID NO: 5 - protein, 4 aa, synthetic construct
DDPK

SEQ ID NO: 6 - protein, 4 aa, synthetic construct
ENDP

SEQ ID NO: 7 - protein, 4 aa, synthetic construct
EDNP

SEQ ID NO: 8 - protein, 4 aa, synthetic construct
DEPN

SEQ ID NO: 9 - protein, 4 aa, synthetic construct
EDLP

SEQ ID NO: 10 - protein, 4 aa, synthetic construct
DEPL

SEQ ID NO: 11 - protein, 4 aa, synthetic construct
DELP

SEQ ID NO: 12 - protein, 4 aa, synthetic construct
DPNE

SEQ ID NO: 13 - protein, 4 aa, synthetic construct
DKPD

SEQ ID NO: 14 - protein, 4 aa, synthetic construct
DPLE